(19) United States
(12) Patent Application Publication
(10) Pub. No.: US 2006/0029661 A1
(43) Pub. Date: Feb. 9, 2006

(54) Orally administrable pharmaceutical formulation

(76) Inventors: Ramachandran Radhakrishnan, Bangalore (IN); Nehru Babu Gaddipati, Somerset, NJ (US); Venkat Subramanian Iyer, Bangalore (IN); Shivaraj Basavaraj Katageri, Bangalore (IN); Prashant Kumar Chakrabarti, Bangalore (IN)

Correspondence Address: KNOBBE MARTENS OLSON & BEAR LLP, 2040 MAIN STREET, FOURTEENTH FLOOR, IRVINE, CA, 92614, US

(21) Appl. No.: 11/199,565

(22) Filed: Aug. 8, 2005

Related U.S. Application Data
(63) Continuation-in-part of application No. 10/096,564, filed on Mar. 13, 2002, now Pat. No. 6,926,906.

(30) Foreign Application Priority Data
Feb. 20, 2002 (IN) IN129/DEL/2002

(51) Int. Cl. A61K 9/64; A61K 31/137; A61K 36/48

(52) U.S. Cl. 424/456; 424/757; 514/649

(57) ABSTRACT

The present invention relates to pharmaceutical formulations for oral administration through a soft gelatin capsule, wherein the pharmaceutical dosage form has pseudoephedrine hydrochloride as the active pharmaceutical ingredient. The active pharmaceutical ingredient, pseudoephedrine hydrochloride as an active is embedded in a suitable matrix, wherein said matrix composition is characterized by reducing the extractability of the pseudoephedrine hydrochloride.

### RELATED APPLICATION INFORMATION

This application is a continuation-in-part of U.S. patent application Ser. No. 10/096,564, filed Mar. 13, 2002, now U.S. Pat. No. 6,925,906 issued Aug. 9, 2005, which claims priority under 35 U.S.C. § 119 (a)-(d) to Indian Patent Application No. IN129/del/2002, filed Feb. 20, 2002.

### FIELD OF THE INVENTION

This invention in general relates to orally administrable pharmaceutical formulations comprising pseudoephedrine hydrochloride. More particularly the present invention provides a pharmaceutical formulation in a soft gelatin capsule comprising pseudoephedrine hydrochloride as an active in a suitable matrix in a manner to reduce the extraction of said active.

### BACKGROUND OF THE INVENTION

Amphetamines have potentially lethal stimulant effects on the central nervous system and heart and are among the most frequently abused drugs. Methamphetamine is the most prevalent synthetic drug manufactured in the United States and is easily produced in clandestine laboratories using commonly used cold remedy products containing pseudoephedrine. Therefore it's important to develop pseudoephedrine formulations that have minimal potential for abuse which is accomplished by minimizing the extractability of pseudoephedrine.

Pseudoephedrine hydrochloride is a vasoconstrictor, which produces vasoconstriction by stimulating (alpha)-receptors within the mucous of the respiratory tract. Clinically pseudoephedrine shrinks the swollen mucous membranes, reduces tissue hyperemia, edema and nasal congestion, and increases nasal airway patency. Its use is therefore significant in the relief from nasal congestion.

Pseudoephedrine hydrochloride tablets and other combination formulations containing pseudoephedrine are used for the temporary relief of nasal congestion caused by common cold. These cold remedies are commercially available as over the counter (OTC) product and contain pseudoephedrine in combination with expectorants and cough suppressants. However, soft gelatin formulations containing only pseudoephedrine Hydrochloride alone as an active ingredient are not commercially available. The following table contains details of commercially available soft gelatin formulations comprising pseudoephedrine hydrochloride in combination with antihistamines and/or analgesics.

| Active Ingredient/s | |
|---|---|
| (Each Capsule contains) | Brand Name/Manufacturer |
| Guaifenesin 200 mg | Robitussin Cold & Cough/ |
| Pseudoephedrine hydrochloride 30 mg | A. H. Robins |
| Dextromethorphan HBr 10 mg | |
| Pseudoephedrine hydrochloride 30 mg | Nyquil/ |
| Doxylamine succinate 6.25 mg | Proctor & Gamble |
| Dextromethorphan HBr 10 mg | |
| Acetaminophen 200 mg | |
| NPseudoephedrine hydrochloride | Dayquil/ |
| 30 mg | Proctor & Gamble |
| Dextromethorphan HBr 10 mg | |
| Acetaminophen 200 mg | |
| Pseudoephedrine hydrochloride 30 mg | Alka-Seltzer Plus |
| Doxylamine succinate 6.25 mg | Night-Time Cold Medicine |
| Dextromethorphan HBr 10 mg | Bayer |
| Acetaminophen 325 mg | |
| Pseudoephedrine hydrochloride 30 mg | Alka-Seltzer Plus |
| Chlorpheniramine Maleate 2 mg | Cold & Cough Medicine |
| Dextromethorphan HBr 10 mg | Bayer |
| Acetaminophen 325 mg | |
| Pseudoephedrine hydrochloride 30 mg | Alka-Seltzer Plus |
| Chlorpheniramine Maleate 2 mg | Cold & Cough Medicine |
| Acetaminophen 325 mg | Bayer |
| Pseudoephedrine hydrochloride 30 mg | Alka-Seltzer Plus |
| Acetaminophen 325 mg | Cold & Sinus Medicine |
| | Bayer |
| Pseudoephedrine hydrochloride 30 mg | Alka-Seltzer Plus |
| Dextromethorphan HBr 10 mg | Cold & Cough Medicine |
| Acetaminophen 325 mg | Bayer |

U.S. Pat. No. 5,409,907 to Blase et al. describes a pharmaceutical suspension comprising a therapeutic amount of pharmaceutical active selected from the group consisting of acetaminophen, famotidine, pseudoephedrine hydrochloride, chlorpheniramine maleate, astemizole, dextromethorphan hydrobromide, guaifenesin, diphenhydramine hydrochloride, loperamide hydrochloride, simethicone, antacids, and combinations thereof. However, the suspending system described therein comprises an effective amount of xanthan gum and microcrystalline cellulose as the suspension medium and suspending agent.

A composition including soybean oil, yellow beeswax and lecithin has been disclosed in the U.S. Pat. No. 6,309,667 to Horvath et al. This disclosure does not address pseudoephedrine hydrochloride as an ingredient in combination with the other excipients.

U.S. Pat. No. 5,175,002 is addressed at a suspension formulation comprising soybean oil, lecithin and wax. However the active in this formulation is Amantidine hydrochloride.

U.S. Pat. No. 5,112,602 to Beurline et al. discloses an oral pharmaceutical liquid suspension comprised of theophylline as the active agent, silicon dioxide, a wetting agent and a hydrocolloid gum.

### SUMMARY OF THE INVENTION

In accordance with one preferred embodiment there is provided an orally administrable pharmaceutical formulation of a matrix composition characterized in that it reduces the extractability of the pseudoephedrine hydrochloride and helps to minimize the abuse potential. The said matrix consists essentially of an active pharmaceutical ingredient embedded into an oily matrix; viscosity imparting agents; surfactant; suspending agent; and suspension medium and a hydrophilic vehicle comprising mixture of glycols.

It has been found that patient compliance is improved if a soft gelatin capsule is used for drug administration, because of its soft, elastic character, which makes it easier to swallow when compared to conventional tablets or hard gelatin capsules. Furthermore, since the dosage form is generally swallowed without chewing, it is unnecessary to flavor or otherwise mask any unpleasant taste of the active pharmaceutical ingredients. Finally, unlike tablets, soft gelatin capsules do not chip or powder. Accordingly, we sought to devise a soft gelatin capsule formulation of pseudoephedrine hydrochloride because of these and other reasons.

In accordance with one preferred embodiment there are provided soft gelatin capsules of a pharmaceutical formulation consisting essentially of about 15-60 mg by weight of pseudoephedrine hydrochloride, about 10-20 mg by weight of yellow beeswax, about 15-25 mg by weight of partially hydrogenated vegetable oil, about 2-8 mg by weight of lecithin, about 2-8 mg by weight of silicon dioxide and about 150-250 mg by weight of soybean oil.

In accordance with another preferred embodiment there are provided methods of making a pharmaceutical formulation comprising preparing a oily blend comprising a soybean oil and partially hydrogenated vegetable oil, heat treating the oily blend with beeswax, wherein the beeswax melts into the oily blend to form an oily liquid matrix, blending lecithin into said oily liquid matrix, mixing pseudoephedrine hydrochloride to said matrix to form a suspension of the pseudoephedrine hydrochloride with the matrix, adding colloidal silicon dioxide to the matrix, and disposing the resultant pharmaceutical complex into a capsule, wherein said orally administrable pharmaceutical is in a liquid form within the capsule.

In accordance with yet another embodiment of the present invention, there is provided an orally administrable pharmaceutical formulation into a soft gelatin capsule consisting essentially of pseudoephedrine hydrochloride as an active ingredient and calcium hydrogen phosphate (CaHPO₄) as a bulking agent embedded into a matrix, wherein the matrix comprises partially hydrogenated vegetable oil and colloidal silicon dioxide as a viscosity-imparting agents; lecithin as a surfactant, yellow beeswax as a suspending agent, soybean oil as a suspension medium and also a mixture of hydrophilic vehicles comprising polyethylene glycol 400, propylene glycol and glycerin which functions to further reduce the extractability of the active from the formulation.

In accordance with another preferred embodiment there is provided an orally administrable pharmaceutical formulation into a soft gelatin capsule consisting essentially of about 30 mg by weight of pseudoephedrine hydrochloride, about 10 to 40 mg by weight of calcium hydrogen phosphate, about 2.0 to 10 mg by weight of yellow beeswax, about 2.0 to 10 mg by weight of partially hydrogenated vegetable oil, about 1.0 to5.0 mg by weight of soy lecithin, about 1.0 to 5.0 mg by weight of colloidal silicon dioxide and about 30 to 70 mg by weight of soybean oil, about 4.0 to 8.0 mg by weight of propylene glycol, about 8.0 to 15 mg by weight of polyethylene glycol 400, about 2.0 to 4.0 mg by weight of glycerin.

In accordance with another preferred embodiment there is provided an orally administrable pharmaceutical formulation into a soft gelatin capsule consisting essentially of about 60 mg by weight of pseudoephedrine hydrochloride, about 20 to 80 mg by weight of calcium hydrogen phosphate, about 4.0 to 20 mg by weight of yellow beeswax, about 4.0 to 20 mg by weight of partially hydrogenated vegetable oil, about 2.0 to10.0 mg by weight of lecithin, about 2.0 to 10.0 mg by weight of colloidal silicon dioxide and about 60 to 140 mg by weight of soybean oil, about 8.0 to 16.0 mg by weight of propylene glycol, about 16.0 to 30 mg by weight of polyethylene glycol 400, about 4.0 to 8.0 mg by weight of glycerin.

In accordance with still another embodiment there is provided a process for preparing an orally administrable pharmaceutical formulation in a soft gelatin capsule comprising, preparing oily blend comprising soybean oil and partially hydrogenated vegetable oil, heat treating the oily blend with beeswax, wherein the beeswax melts into the oily blend to form an oily liquid matrix, dispersing colloidal silicon dioxide in oily matrix, adding lecithin, polyethylene glycol 400, propylene glycol and glycerin, sifting of calcium hydrogen phosphate and pseudoephedrine hydrochloride, adding sifted calcium hydrogen phosphate and pseudoephedrine hydrochloride to above matrix with continuous stirring, mixing the resultant to get uniform suspension, and disposing the resultant pharmaceutical complex into a capsule, wherein said orally administrable pharmaceutical formulation is in a liquid form within the capsule.

In accordance with yet another embodiment of the present invention, there is provided an orally administrable pharmaceutical formulation into a soft gelatin capsule consisting essentially of pseudoephedrine hydrochloride as an active ingredient embedded into a matrix, wherein the matrix consisting essentially of polyethylene glycol 400, propylene glycol, glycerin and polyvinylpyrrolidone.

In accordance with yet another embodiment of the present invention, there is provided an orally administrable pharmaceutical formulation into a soft gelatin capsule consisting essentially of about 30 mg by weight of pseudoephedrine hydrochloride, about 220 to 300 mg by weight of polyethylene glycol 400, about 25.0 to 35.0 mg by weight of propylene glycol, about 2.0 to 5.0 mg by weight of glycerin and about 12.0 to 30.0 mg by weight of polyvinylpyrrolidone.

In accordance with yet another embodiment of the present invention, there is provided an orally administrable pharmaceutical formulation into a soft gelatin capsule consisting essentially of about 60 mg by weight of pseudoephedrine hydrochloride, about 440 to 600 mg by weight of polyethylene glycol 400, about 50.0 to 70.0 mg by weight of propylene glycol, about 4.0 to 10.0 mg by weight of glycerin and about 24.0 to 60.0 mg by weight of polyvinylpyrrolidone.

In accordance with one other embodiment of the present invention there is provided a process to produce a pharmaceutical formulation in a soft gelatin capsule comprising, heating the mixture of polyethylene glycol 400, propylene glycol and glycerin, adding polyvinylpyrrolidone in mixture with continuous stirring to get clear solution, adding pseudoephedrine hydrochloride in above mixture with continuous stirring to get clear solution, and disposing the resultant into a capsule, wherein said orally administrable pharmaceutical is in a liquid form within the capsule.

In accordance with yet another embodiment of the present invention, there is provided an orally administrable pharmaceutical formulation into a soft gelatin capsule consisting essentially of pseudoephedrine hydrochloride as an active ingredient embedded into a matrix, wherein the matrix consists essentially of polyethylene glycol 400, glycerin, polyoxyl 35 hydrogenated castor oil and polyethylene glycol 4000.

In accordance with yet another embodiment of the present invention, there is provided an orally administrable pharmaceutical formulation into a soft gelatin capsule consisting essentially of about 30 mg by weight of pseudoephedrine hydrochloride, about 75.0 to 100 mg by weight of polyethylene glycol 400, about 2.0 to 4.0 mg by weight of glycerin, about 4.0 to 10 mg by weight of polyoxyl 35 hydrogenated castor oil and about 3.0 to 10 mg by weight of polyethylene glycol 4000.

In accordance with one other embodiment of the present invention, there is provided an orally administrable pharmaceutical formulation into a soft gelatin capsule consisting essentially of about 60 mg by weight of pseudoephedrine hydrochloride, about 150 to 200 mg by weight of polyethylene glycol 400, about 4.0 to 8.0 mg by weight of glycerin, about 8.0 to 20 mg by weight of polyoxyl 35 hydrogenated castor oil and about 6.0 to 20 mg by weight of polyethylene glycol 4000.

In accordance with another embodiment of the present invention, there is provided a process for preparing an orally administrable soft gelatin capsule comprising, warming the mixture of polyethylene glycol 400, glycerin & polyoxyl 35 hydrogenated castor oil, adding polyethylene glycol 4000 with continuous stirring, adding pseudoephedrine hydrochloride in above mixture with continuous stirring to get uniform dispersion and disposing the resultant into a capsule, wherein said orally administrable pharmaceutical formulation is in a liquid form within the capsule.

In accordance with yet another embodiment of the present invention, there is provided an orally administrable pharmaceutical formulation into a soft gelatin capsule consisting essentially of pseudoephedrine hydrochloride as an active ingredient embedded into a matrix, wherein the matrix consists essentially of polyethylene glycol 400, glycerin, polyoxyl 35 hydrogenated castor oil, glyceryl monostearate and polyethylene glycol 4000.

In accordance with yet another embodiment of the present invention, there is provided an orally administrable pharmaceutical formulation into a soft gelatin capsule consisting essentially of about 30 mg by weight of pseudoephedrine hydrochloride about 75.0 to 100 mg by weight of polyethylene glycol 400, about 2.0 to 4.0 mg by weight of glycerin, about 4.0 to 10 mg by weight of polyoxyl 35 hydrogenated castor oil, about 4.0 to 10 mg by weight of glyceryl monostearate and about 3.0 to 10 mg by weight of polyethylene glycol 4000.

In accordance with one other embodiment of the present invention, there is provided an orally administrable pharmaceutical formulation into a soft gelatin capsule consisting essentially of about 60 mg by weight of pseudoephedrine hydrochloride about 150 to 200 mg by weight of polyethylene glycol 400, about 4.0 to 8.0 mg by weight of glycerin, about 8.0 to 20 mg by weight of polyoxyl 35 hydrogenated castor oil, about 8.0 to 20 mg by weight of glyceryl monostearate and about 6.0 to 20 mg by weight of polyethylene glycol 4000.

In accordance with another embodiment of the present invention, there is provided a process for preparing an orally administrable soft gelatin capsule comprising, mixing & warming polyethylene glycol 400, propylene glycol, glycerin & polyoxyl 35 hydrogenated castor oil, adding glyceryl monostearate and polyethylene glycol 4000 in above mixture with continuous stirring, adding pseudoephedrine hydrochloride in above mixture with continuous stirring to get uniform suspension.

One possible advantage of preferred embodiments is that the active ingredient (either alone or along with one or more excipients) is coated with wax, making the extraction of pseudoephedrine and its derivatives more difficult. Yet another advantage of the preferred embodiments is that the drug delivery of the pharmaceutical formulation is achieved by a soft gelatin capsule and this makes it relatively difficult for someone to extract the pharmaceutically active ingredient, unlike the case of a tablet as an OTC drug product. In addition, in comparison to all commercial soft gelatin capsule formulations described in this document containing pseudoephedrine hydrochloride as a solution, the formulation containing wax described in this embodiment makes the extraction process relatively difficult. Hence the possibility of using the softgel product for the abuse is minimized.

Another possible advantage of preferred embodiments is that preferred formulations include excipients like yellow beeswax and soybean oil, which are natural substances that make the extraction further difficult. This, in conjunction with the soft gelatin encapsulation, makes it relatively a complex multi-step process to extract pseudoephedrine from the oily matrix. Thus the preferred embodiments considerably minimize the potential to abuse the drug product.

Further the extractability of pseudoephedrine from the formulation with improved matrix comprising hydrophilic vehicles containing mixture of glycols is about 11% which is lower than all conventional dosage forms available.

### DETAILED DESCRIPTION OF THE INVENTION

The present invention relates to pharmaceutical formulations having pseudoephedrine hydrochloride as the pharmaceutically active ingredient for oral administration in the form of soft gelatin capsules. The formulation also comprises partially hydrogenated vegetable oil, yellow beeswax, colloidal silicon dioxide, soybean oil and lecithin. In preferred embodiments, we have used soybean oil as a suspension medium and yellow beeswax as a suspending agent. Hydrogenated vegetable oil has been used as a viscosity inducing agent and colloidal silicon dioxide is used to achieve uniform dose dispersion in preferred embodiments. In a preferred embodiment, the capsules do not contain any pharmaceutically active materials other than Pseudoephedrine and/or a salt thereof.

The phrase “consisting essentially of” is used herein in its ordinary sense, including that the recited composition may further include ingredients which do not materially affect the basic and novel properties of the composition, including, but not limited to, additives such as colorants and flavorants.

Propylene glycol is used in wide variety of pharmaceutical formulations and is generally regarded as a nontoxic material. It is used as solvent, antimicrobial preservative, disinfectant, humectant, plasticizer, water-miscible cosolvent and stabilizer for vitamins. Propylene Glycol is a clear, colorless, viscous, practically odorless liquid with a sweet, slightly acrid taste resembling glycerin. It is official in British Pharmacopoeia and USP. Propylene glycol is used in a wide variety of pharmaceutical formulations and is generally regarded as a nontoxic material. Based on metabolic and toxicological data, the WHO has set an acceptable daily intake of propylene glycol at up to 25 mg/kg body weight. (Ref: Handbook of Pharmaceutical Excipients, 2^{nd} edition, P. No: 407-408)

Inclusion of Polyethylene Glycol 400 was found to be useful. Polyethylene Glycols can be used to enhance the aqueous solubility or dissolution characteristics of poorly soluble drugs. Polyethylene glycols are also called as Macrogols. Macrogols are relatively stable, non-toxic compounds, which have a range of properties depending on their molecular weight. They are widely used in pharmaceutical manufacturing as water soluble bases for topical preparations and suppositories, as solvents and vehicles, and as solubilizing agents, tablet binders, plasticizers in film coating, and tablet lubricants. They have also been reported to have antibacterial properties. (Ref: Martindale, The Complete Drug Reference—33^{rd} edition, P. No. 1630)

Polyvinylpyrrolidone (PVP K-30) has been used in a variety of Pharmaceutical formulations. It has a property of increasing viscosity and an ability to increase solubility of poorly soluble active drugs. It is used in this formulation to enhance the solubility of poorly aqueous soluble drugs and to prevent the recrystallization. (Ref: Handbook of Pharmaceutical Excipients, 2^{nd} edition, P. No: 392)

Glyceryl monostearate is hard, waxy mass or unctuous powder or flakes, white or almost white, practically insoluble in water, soluble in alcohol at 60° C. Glyceryl monostearate is used in this formulation as a viscosity imparting agent to prevent the settling of pharmaceutical active ingredients. (Reference: European Pharmacopoeia, 5^{th} Edition, Volume 2, P. No: 1677)

Glycerin is a syrupy liquid, unctuous to touch, colorless or almost colorless, clear, very hygroscopic, miscible with water and with alcohol, slightly soluble in acetone, practically insoluble in fatty oils and in essential oils. Glycerin is used as cosolvent in this formulation to enhance/improve the solubility of drug substances. (Reference: European Pharmacopoeia, 5^{th} Edition, Volume 2, P. No: 1671)

Polyoxyl 35 Hydrogenated castor oil: It contains mainly trihydroxystearyl glycerol ethoxylated with 7 to 60 molecules of ethylene oxide (nominal value), with small amount of macrogol hydroxystearate and of the corresponding free glycol. It results from the reaction of hydrogenated castor oil with less than 10 units of ethylene oxide per molecule, is a yellowish, turbid, viscous liquid, practically insoluble in water, dispersible in alcohol, and soluble in acetone. Polyoxyl hydrogenated castor oil with more than 20 units of ethylene oxide per molecule is a white or yellowish, semi-liquid or pasty mass, freely soluble in water, in alcohol, and in acetone; practically insoluble in petroleum spirit. Polyoxyl castor oils are macrogol esters used as emulsifying and solubilzing agents. (Reference: Martindale, 33^{rd} edition, P. No: 1347-2)

Polyethylene glycol 4000 (PEG 4000): Polyethylene glycols are condensation polymers of ethylene oxide and water & are also called as Macrogols. Each macrogol's name is followed by a number indicating its approximate average molecular weight; thus macrogol 4000 (PEG 4000) has an average molecular weight of about 4000. Macrogols with an average molecular weight of 200 to 600 are clear to slightly hazy, colorless or almost colorless, viscous liquid with a slight characteristic odor; those with an average molecular weight of more than 1000 are white to off-white solids, also with a slight characteristic odor, which vary in consistency between soft unctuous pastes and hard waxy flakes, beads, or powder. Viscosity increases with increasing molecular weight, but hygroscopic nature decreases and, at average molecular weights above 4000, hygroscopic characteristic is low. (Reference: Martindale, 33^{rd} edition, P. No: 1630-1)

According to a preferred embodiment, wax forms part of the fill composition that is inside the gelatin shell. A coating of the pharmaceutically active product in wax and oil mixture is achieved making it difficult to isolate the active from the formulation.

Although, the parent application addresses the novelty of using specific matrix composition in a soft gelatin dosage form to minimize the extractability of pseudoephedrine hydrochloride there still remains the necessity to quantify the extractability and improve upon it, if possible. Almost all the pseudoephedrine (97%) was extractable from the commercial pseudoephedrine tablets. The extraction procedure employed is detailed in paragraph [0063]. The same extraction procedure was utilized to determine the extractability of pseudoephedrine from softgel formulations shown below in various embodiments.

Accordingly, further development trials were continued and we found drastic improvement in the formulation with respect to decrease in extractability of pseudoephedrine hydrochloride from the parent formulation, the details of various compositions and the extent of extractability is presented for each embodiment.

The following examples illustrate the preferred embodiments of pharmaceutical compositions comprising pseudoephedrine hydrochloride as principal ingredient.

### EXAMPLES

The examples presented below provide a preferred amount of each component or a range of preferred amounts of each component that is present in a single dosage according to preferred embodiments. To make multiple dosages or multiple capsules, the amounts are multiplied by a scaling factor corresponding to the number of doses desired.

### Example 1

| Ingredients | Composition by weight | |
|---|---|---|
| Pseudoephedrine hydrochloride | 15-60 | mg |
| Yellow Beeswax | 10-20 | mg |
| Partially Hydrogenated Vegetable Oil | 15-25 | mg |
| Lecithin | 2-8 | mg |
| Colloidal Silicon Dioxide | 2-8 | mg |
| Soybean Oil, | 150-250 | mg |

In Example 1 above, the soybean oil and partially hydrogenated vegetable oil were mixed to form an oily blend, the beeswax was added to the oily blend and the mixture heated to a temperature sufficient to melt the beeswax into the oily blend, but not so high as to degrade the oils (preferably from about 75 to 85° C.), thereby forming an oily matrix. The oily blend may be heated either before or after the addition of the beeswax. Colloidal silicon dioxide was then dispersed in the oily matrix, followed by addition of the lecithin. Pseudoephedrine hydrochloride was sifted or provided as a fine powder and added to the oily matrix with continuous stirring to form a generally uniform suspension. The suspension was then disposed into gelatin capsules. The suspension was in liquid form when placed into the capsules and remained a liquid afterwards within the capsule.

A composition according to Example 1 was made and filled into several gelatin capsules where each capsule was filled with 147.5 mg of a composition consisting of 30 mg of pseudoephedrine HCl, 10 mg partially hydrogenated vegetable oil, 7.5 mg yellow beeswax, 2.5 mg soya lecithin, 2.5 mg colloidal silicon dioxide and 95 mg soybean oil. The capsules were then subjected to the extraction procedure set forth in paragraph [0063] below, and the extractability of the pseudoephedrine HCl was found to be 40%.

### Example 2

| Ingredients | Composition by weight | |
|---|---|---|
| Pseudoephedrine hydrochloride | 30 | mg |
| Yellow Beeswax | 2.0-10 | mg |
| Partially Hydrogenated Vegetable Oil | 2.0-10 | mg |
| Lecithin | 1.0-5.0 | mg |
| Colloidal Silicon Dioxide | 1.0-5.0 | mg |
| Soybean Oil | 30-70 | mg |
| Propylene Glycol | 4.0-8.0 | mg |
| Polyethylene glycol 400 | 8.0-15.0 | mg |
| Glycerin | 2.0-4.0 | mg |
| Calcium hydrogen phosphate | 10-40 | mg |

### Example 3

| Ingredients | Composition by weight | |
|---|---|---|
| Pseudoephedrine hydrochloride | 60 | mg |
| Yellow Beeswax | 4.0-20 | mg |
| Partially Hydrogenated Vegetable Oil | 4.0-20 | mg |
| Lecithin | 2.0-10.0 | mg |
| Colloidal Silicon Dioxide | 2.0-10.0 | mg |
| Soybean Oil | 60-140 | mg |
| Propylene Glycol | 8.0-16.0 | mg |
| Polyethylene glycol 400 | 16.0-30.0 | mg |
| Glycerin | 4.0-8.0 | mg |
| Calcium hydrogen phosphate | 20-80 | mg |

In Examples 2 and 3 above, the soybean oil and partially hydrogenated vegetable oil were mixed to form an oily blend, the beeswax was added to the oily blend and the mixture heated to a temperature sufficient to melt the beeswax into the oily blend, but not so high as to degrade the oils (preferably from about 75 to 85° C.), thereby forming an oily matrix. The oily blend may be heated either before or after the addition of the beeswax. Colloidal silicon dioxide was then dispersed in the oily matrix, followed by addition of the lecithin, polyethylene glycol 400, propylene glycol and glycerin into the matrix. Calcium hydrogen phosphate and pseudoephedrine hydrochloride were sifted or provided as a fine powder and added to the oily matrix with continuous stirring to form a generally uniform suspension. The suspension was then disposed into gelatin capsules. The suspension was in liquid form when placed into the capsules and remained a liquid afterwards within the capsule.

A composition according to Example 2 was made and filled into several gelatin capsules where each capsule was filled with 125 mg of a composition consisting of 30 mg of pseudoephedrine HCl, 2.5 mg partially hydrogenated vegetable oil, 2.5 mg yellow beeswax, 1.25 mg soya lecithin, 1.25 mg colloidal silicon dioxide, 40 mg soybean oil, 5 mg propylene glycol, 10 mg polyethylene glycol 400 (PEG 400), 2.5 mg glycerin, and 30 mg calcium hydrogen phosphate. The capsules were then subjected to the extraction procedure set forth in paragraph [0063] below, and the extractability of the pseudoephedrine HCl was found to be 20%.

### Example 4

| Ingredients | Composition by weight | |
|---|---|---|
| Pseudoephedrine hydrochloride | 30.0 | mg |
| Propylene Glycol | 25.0-35.0 | mg |
| Polyethylene glycol 400 | 220.0-300.0 | mg |
| Glycerin | 2.0-5.0 | mg |
| Polyvinyl pyrrolidone (PVP K-30) | 12.0-30.0 | mg |

### Example 5

| Ingredients | Composition by weight | |
|---|---|---|
| Pseudoephedrine hydrochloride | 60.0 | mg |
| Propylene Glycol | 50.0-70.0 | mg |
| Polyethylene glycol 400 | 440.0-600.0 | mg |
| Glycerin | 4.0-10.0 | mg |
| Polyvinyl pyrrolidone (PVP K-30) | 24.0-60.0 | mg |

In Examples 4 and 5 above, the polyethylene glycol, propylene glycol and glycerin were mixed and heated or warmed, preferably to a temperature of about 65 to 75° C. Polyvinylpyrrolidone was then added to the mixture with continuous stirring to get a substantially clear solution. Pseudoephedrine hydrochloride was then added to the solution with continuous stirring to get clear solution. The solution was then placed into gelatin capsules. The solution was in liquid form when placed into the capsules and remained a liquid afterwards within the capsule.

A composition according to Example 4 was made and filled into several gelatin capsules where each capsule was filled with 300 mg of a composition consisting of 30 mg of pseudoephedrine HCl, 27.5 mg propylene glycol, 225 mg PEG 400, 2.5 mg glycerin, and 15 mg polyvinyl pyrrolidone (PVP K-30). The capsules were then subjected to the extraction procedure set forth in paragraph [0063] below, and the extractability of the pseudoephedrine HCI was found to be 11%.

### Example 6

| Ingredients | Composition by weight | |
|---|---|---|
| Pseudoephedrine hydrochloride | 30 | mg |
| Polyethylene glycol 400 | 75-100 | mg |
| Polyethylene glycol 4000 | 3.0-10 | mg |
| Glycerin | 2.0-4.0 | mg |
| Cremophor EL 35 (Polyoxyl 35 hydrogenated | 4.0-10 | mg |
| castor oil) | | |

### Example 7

| Ingredients | Composition by weight | |
|---|---|---|
| Pseudoephedrine hydrochloride | 60 | mg |
| Polyethylene glycol 400 | 150-200 | mg |
| Polyethylene glycol 4000 | 6.0-20 | mg |
| Glycerin | 4.0-8.0 | mg |
| Cremophor EL 35 (Polyoxyl 35 hydrogenated | 8.0-20 | mg |
| castor oil) | | |

In Examples 6 and 7 above, the polyethylene glycol, glycerin and polyoxyl 35 hydrogenated castor oil were mixed and heated or warmed, preferably to a temperature of about 65 to 75° C. The polyethylene glycol 4000 was then added with continuous stirring followed by the pseudoephedrine hydrochloride with continuous stirring to provide a uniform dispersion or suspension. The dispersion was then placed into gelatin capsules. The dispersion was in liquid form when placed into the capsules and remained a liquid afterwards within the capsule.

A composition according to Example 6 was made and filled into several gelatin capsules where each capsule was filled with 125 mg of a composition consisting of 30 mg of pseudoephedrine HCl, 82.5 mg PEG 400, 5 mg of PEG 4000, 2.5 mg glycerin, and 5 mg Cremophor EL 35. The capsules were then subjected to the extraction procedure set forth in paragraph [0063] below, and the extractability of the pseudoephedrine HCl was found to be 18.3%.

A composition according to Example 7 was made and filled into several gelatin capsules where each capsule was filled with 250 mg of a composition consisting of 60 mg of pseudoephedrine HCl, 165 mg PEG 400, 10 mg of PEG 4000, 5 mg glycerin, and 10 mg Cremophor EL 35. The capsules were then subjected to the extraction procedure set forth in paragraph [0063] below, and the extractability of the pseudoephedrine HCl was found to be 15.5%.

### Example 8

| Ingredients | Composition by weight | |
|---|---|---|
| Pseudoephedrine hydrochloride | 30 | mg |
| Polyethylene glycol 400 | 75-100 | mg |
| Polyethylene glycol 4000 | 3.0-10 | mg |
| Glycerin | 2.0-4.0 | mg |
| Glyceryl monosterate | 4.0-10 | mg |
| Cremophor EL 35 (Polyoxyl 35 hydrogenated | 4.0-10 | mg |
| castor oil) | | |

### Example 9

| Ingredients | Composition by weight | |
|---|---|---|
| Pseudoephedrine hydrochloride | 60 | mg |
| Polyethylene glycol 400 | 150-200 | mg |
| Polyethylene glycol 4000 | 6.0-20 | mg |
| Glycerin | 4.0-8.0 | mg |
| Glyceryl monosterate | 8.0-20 | mg |
| Cremophor EL 35 (Polyoxyl 35 hydrogenated | 8.0-20 | mg |
| castor oil) | | |

In Examples 8 and 9 above, the polyethylene glycol, glycerin and polyoxyl 35 hydrogenated castor oil were mixed and heated or warmed, preferably to a temperature of about 65 to 75° C. The glyceryl monostearate and polyethylene glycol 4000 were then added with continuous stirring followed by the pseudoephedrine hydrochloride with continuous stirring to provide a uniform suspension or dispersion. The suspension was then placed into gelatin capsules. The suspension was in liquid form when placed into the capsules and remained a liquid afterwards within the capsule.

A composition according to Example 8 was made and filled into several gelatin capsules where each capsule was filled with 125 mg of a composition consisting of 30 mg of pseudoephedrine HCl, 77.5 mg PEG 400, 5 mg of PEG 4000, 2.5 mg glycerin, 5 mg glycerol monostearate, and 5 mg Cremophor EL 35. The capsules were then subjected to the extraction procedure set forth in paragraph [0063] below, and the extractability of the pseudoephedrine HCl was found to be 14.3%.

A composition according to Example 9 was made and filled into several gelatin capsules where each capsule was filled with 250 mg of a composition consisting of 60 mg of pseudoephedrine HCl, 155 mg PEG 400, 10 mg of PEG 4000, 5 mg glycerin, 10 mg glycerol monostearate, and 10 mg Cremophor EL 35. The capsules were then subjected to the extraction procedure set forth in paragraph [0063] below, and the extractability of the pseudoephedrine HCl was found to be 16.3%.

Although pseudoephedrine hydrochloride is a preferred form of the active, use of the free base or other salts of pseudoephedrine, or combinations thereof, is also contemplated.

In general, gelatin capsule formulations for soft gelatin capsule comprise gelatin, plasticizer, solvent and optional ingredients such as flavors and colorants. Typically the plasticizer includes glycerin, Anidrisorb or sorbitol. A preferred plasticizer in this case is glycerin. One preferred gelatin formulation for the soft gelatin capsule used in accordance with preferred embodiments includes gelatin in the range of about 40-45% by weight and a plasticizer in the range of about 15-25% by weight. Capsule formulation can also include other suitable additives, which impart specific characteristics such as the look and feel of the capsule.

The following examples illustrate preferred embodiments of several soft-gelatin-shell pseudoephedrine hydrochloride formulations. Capsules may be made from the following formulations by methods that include those well known in the pharmaceutical art. Capsules made by other methods or by using different formulations are also contemplated for use with the pharmaceutical formulations and methods described herein. These examples illustrate particular embodiments of the invention and are not intended to limit the scope of the invention in any way.

### Example 10

| Ingredient | Percentage by weight |
|---|---|
| Gelatin | 43.4% |
| Glycerin | 20% |
| Water | 36.6% |

### Example 11

| Ingredient | Percentage by weight |
|---|---|
| Gelatin | 45% |
| Sorbitol 70% solution | 18% |
| Water | 37% |

### Example 12

| Ingredient | Percentage by weight |
|---|---|
| Gelatin | 42% |
| Anidrisorb 85/70 or Polysorb 85/70 | 25% |
| Water | 33% |

### Example 13

| Ingredient | Percentage by weight |
|---|---|
| Gelatin | 42% |
| Glycerin | 16% |
| Sorbitol 70% solution | 4% |
| Water | 38% |

The capsules as manufactured above may be provided as either coated or uncoated. If capsules are coated, they may be coated with any suitable coating including the following coating compositions.

### Example 14

| Ingredient | Percentage by weight |
|---|---|
| Hydroxypropylmethylcellulose 15 cps | 3.2% |
| Plasdone S630 | 0.64% |
| PEG 6000 | 0.64% |
| Isopropyl alcohol | 76.32% |
| Water | 19.2% |

### Example 15

| Ingredient | Percentage by weight |
|---|---|
| HPMC 15 cps | 3.07% |
| Triethyl citrate | 0.307% |
| FD & C Red 40 | 0.294% |
| Isopropyl alcohol | 78.89% |
| Water | 17.43% |

The method used for extraction of pseudoephedrine from pseudoephedrine hydrochloride soft gelatin capsules is as follows:

Sample preparation:

1. Collect enough Soft gelatin capsules that have approximately 480 mg pseudoephedrine hydrochloride.
2. Place the collected samples in a 250 ml separating funnel. Add 100 ml deionized water to soften and rupture the gelatin capsules.
3. Add 10 ml of 1 M NaOH and shake the samples.
4. Allow the samples to sit overnight and allow the gelatin to fully dissolve and tablets to disintegrate.
5. Add 100 ml of hexane and shake the separating funnel vigorously and similar process of extraction is to be repeated two more times with 100 ml of hexane each and extraction fluids will be combined. In addition hexane extracts are passed through anhydrous sodium sulfate washed with hexane and this step will remove any moisture present in hexane to avoid any erroneous values due to traces of moisture carried over via hexane.
6. Allow the samples to remain undisturbed till layers separate.
7. Collect the top hexane layer into a suitable tared beaker and evaporate the hexane under a stream of air.
   Residue Testing (Residue Weight)
1. After the hexane is evaporated, weigh the beaker and residue.
2. Using the weight of the empty beaker from the sample preparation as tare, determine the weight of the residue that was collected.
3. This weight will be used with the assay to determine the total amount of pseudoephedrine that was recovered from each sample.
   Residue Testing (Residue Assay)
1. Use standard Gravimetric or High Performance Liquid Chromatography procedure for the following parameters—equipment, reagents and chromatography conditions. The assay standard will be used to run the samples (150 mg pseudoephedrine hydrochloride into a 500 ml volumetric flask).
2. Residue sample—weigh 100 mg of the residue and transfer to a 100 ml volumetric flask. Add 10 ml of citric acid solution Dilute to volume with water. Mix thoroughly.
3. Assay the sample using Gravimetry method by estimating weight of residue or by carrying out assay analysis by HPLC.
4. Convert results to pseudoephedrine hydrochloride from the sample assay result that is pseudoephedrine (change in molecular weight pseudoephedrine hydrochloride=201.69, pseudoephedrine=165.23)
   Reporting Results:
1. Residue description
2. Residue weight
3. Percent pseudoephedrine hydrochloride
4. Calculate the total amount of pseudoephedrine hydrochloride from the residue that was recovered

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the formulations and methods may be formulated or performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein. For example, equivalents to various components in pharmaceutical compositions such as those used herein are known, including from the US Pharmacopeia and US FDA.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

What is claimed is:

1. An orally administrable pharmaceutical formulation consisting essentially of pseudoephedrine hydrochloride and calcium hydrogen phosphate suspended in an oily matrix, said oily matrix consisting essentially of beeswax as a suspending agent, soybean oil as a suspension medium, lecithin as a surfactant, and colloidal silicon dioxide and/or partially hydrogenated vegetable oil as a viscosity imparting agent along with a mixture of hydrophilic vehicles wherein the formulation is a liquid, and wherein said matrix composition is characterized by reducing the extractability of the pseudoephedrine hydrochloride.

2. The orally administrable pharmaceutical formulation of claim 1, wherein the formulation is contained within a soft gelatin capsule.

3. The orally administrable pharmaceutical formulation of claim 1, wherein the mixture of hydrophilic vehicles comprises polyethylene glycol 400, propylene glycol and glycerin.

4. An orally administrable pharmaceutical formulation consisting essentially of pseudoephedrine hydrochloride suspended in a liquid matrix, consisting essentially of polyethylene glycol, propylene glycol, glycerin, and/or polyvinylpyrrolidone and wherein said matrix composition is characterized by reducing the extractability of the pseudoephedrine hydrochloride.

5. The orally administrable pharmaceutical formulation of claim 4, wherein the formulation is contained within a soft gelatin capsule.

6. An orally administrable formulation consisting essentially of pseudoephedrine hydrochloride suspended in a liquid matrix, said liquid matrix consisting essentially of polyethylene glycol, glycerin, polyoxyl 35 hydrogenated castor oil and/or polyethylene glycol 4000, and wherein said matrix composition is characterized by reducing the extractability of the pseudoephedrine hydrochloride.

7. The orally administrable pharmaceutical formulation of claim 6, wherein the formulation is contained within a soft gelatin capsule.

8. An orally administrable formulation consisting essentially of pseudoephedrine hydrochloride suspended in liquid matrix, said liquid matrix consisting essentially of polyethylene glycol, glycerin, polyoxyl 35 hydrogenated castor oil, glyceryl monostearate and/or polyethylene glycol 4000, and wherein said matrix composition is characterized by reducing the extractability of the pseudoephedrine hydrochloride.

9. The orally administrable pharmaceutical formulation of claim 8, wherein the formulation is contained within a soft gelatin capsule.

10. A process for preparing an orally administrable pharmaceutical formulation of claim 1 comprising:
preparing an oily blend comprising soybean oil and partially hydrogenated vegetable oil;
heat treating the oily blend with beeswax, wherein the beeswax melts into the oily blend to form an oily liquid matrix;
dispersing colloidal silicon dioxide in oily matrix; adding lecithin, and a mixture of hydrophilic vehicles into the same;
sifting of calcium hydrogen phosphate and pseudoephedrine hydrochloride;
adding sifted calcium hydrogen phosphate and pseudoephedrine hydrochloride to resultant matrix with continuous stirring;
mixing the resultant to get uniform suspension, and disposing the resultant pharmaceutical complex into the capsule, wherein said orally administrable pharmaceutical formulation is in a liquid suspension form within the capsule.

11. The process of claim 10, wherein the mixture of hydrophilic vehicles comprises polyethylene glycol 400, propylene glycol and glycerin.

12. A process for preparing an orally administrable pharmaceutical formulation of claim 4 comprising:
heating the mixture of polyethylene glycol, propylene glycol and glycerin;
adding polyvinylpyrrolidone in above mixture with continuous stirring to get clear solution;
adding pseudoephedrine hydrochloride in the resultant mixture with continuous stirring to get clear solution, and
disposing the resultant into the capsule, wherein said orally administrable pharmaceutical formulation is in a liquid form within the capsule.

13. A process for preparing an orally administrable pharmaceutical formulation of claim 6 comprising:
warming the mixture of polyethylene glycol, glycerin and polyoxyl 35 hydrogenated castor oil;
adding polyethylene glycol 4000 with continuous stirring;
adding pseudoephedrine hydrochloride in above mixture with continuous stirring to get uniform dispersion, and
disposing the resultant pharmaceutical complex into the capsule, wherein said orally administrable pharmaceutical formulation is in a liquid form within the capsule.

14. A process for preparing an orally administrable pharmaceutical formulation of claim 8 comprising:
mixing and warming polyethylene glycol, glycerin and polyoxyl 35 hydrogenated castor oil;
adding glyceryl monostearate and polyethylene glycol 4000 in above mixture with continuous stirring;
adding pseudoephedrine hydrochloride in above mixture with continuous stirring to get uniform suspension, and
disposing the resultant into the capsule, wherein said orally administrable pharmaceutical formulation is in a liquid form within the capsule.
